(19) United States
(12) Patent Application Publication
(10) Pub. No.: US 2014/0248655 A1
(43) Pub. Date: Sep. 4, 2014

(54) QUANTITATIVE SOFT AGAR COLONY FORMATION ASSAY USING TETRAZOLIUM THAT GENERATES WATER-SOLUBLE FORMAZAN

(71) Applicant: KAGOSHIMA UNIVERSITY, Kagoshima (JP)

(72) Inventor: Tatsuhiko Furukawa, Kagoshima (JP)

(73) Assignees: NIPPON MEDICAL & CHEMICAL INSTRUMENTS CO., LTD., Osaka-shi, Osaka (JP); KAGOSHIMA UNIVERSITY, Kagoshima-shi, Kagoshima (JP)

(21) Appl. No.: 14/349,397

(22) PCT Filed: Oct. 3, 2012
(86) PCT No.: PCT/JP2012/075598

§ 371 Date: Apr. 3, 2014

(30) Foreign Application Priority Data
Oct. 3, 2011 (JP) 2011-219433

(51) Int. Cl. G01N 33/50

(52) U.S. Cl. CPC G01N 33/5014; G01N 2500/04
USPC 435/29

(57) ABSTRACT

This invention provides a quantitative assay technique for soft agar colony formation that can be carried out rapidly with high accuracy. Specifically, this invention relates to a method for evaluation of cell survival comprising: a step of overlaying agar at the bottom of a vessel with agar containing cells, overlaying the agar containing cells with a medium and culturing the cells; a step of removing the medium, adding tetrazolium that produces water-soluble formazan and an electron carrier and culturing the cells; and a step of evaluating cell survival based on the color developed by the water-soluble formazan.

### TECHNICAL FIELD

The present invention relates to a technique of quantitative assay for soft agar colony formation involving the use of for example, tetrazolium that can produce water-soluble formazan.

### BACKGROUND ART

The established in vitro assay systems are important techniques that have supported progress in cancer research. It has heretofore been known that anchorage-independent growth is highly correlated with carcinogenesis and that neoplastic transformation of cells had been evaluated based on the presence or absence of anchorage-independent growth capacity.

A soft agar colony formation assay is an in vitro assay technique for tumorigenic potentials that can be carried out in a relatively simple manner. A soft agar colony formation assay is carded out by dispensing a soft agar medium into a dish, culturing cells in such soft agar medium, and counting the number of formed colonies so as to evaluate cell growth capacity. According to conventional soft agar colony formation assay techniques, however, it was difficult to perform quantification because of the three-dimensional cell growth that occurs in agar. Generally speaking, available assay techniques were limited to semi-quantitative evaluation of the anchorage-independent growth capacity by means of cell staining in the past.

Accordingly, establishment of a screening method that enables efficient analysis of many specimens within a short period of time by quantitative assay for soft agar colony formation has been awaited.

In contrast, a technique of, for example, CytoSelect™ 96-well in vitro tumor sensitivity assay (soft agar colony formation assay, Cell Biolabs, Inc.) uses a tetrazolium salt (i.e., MTT; 3-[4,5-dimethylthiazol-2-yl]-2,5-diphenyltetrazolium bromide) to quantify cell growth capacity in soft agar colony formation assays. MTT is a yellow aqueous solution, and it is reduced by mitochondrial dehydrogenase in a cell upon incorporation thereof into a viable cell, following, which formazan is formed. Formazan is a blue, water-insoluble crystal, which is precipitated upon its production. When the precipitated formazan is dissolved with a surfactant such as DMSO, a red-purple solution is produced. The absorbance of the solution may then be assayed, so as to quantify cell growth capacity by a colorimetric method.

In accordance with the CytoSelect™ 96-well in vitro tumor sensitivity assay technique, specifically, agar at the bottom of a 96-well microtiter plate is overlaid with soft agar containing cells, the soft agar is further overlaid with a medium containing a drug, such as an anticancer agent, and culture is conducted for approximately 1 week. Thereafter, an agar solubilization buffer is added thereto so as to solubilize agar. The solubilized mixture is transferred to another 96-well microtiter plate, an MTT solution is added, and incubation is carried out for 2 to 4 hours. Thereafter, a surfactant is added thereto, and incubation is carried out for an additional 2 to 4 hours. Subsequently, the absorbance at 570 nm is measured using a microtiter plate reader.

In accordance with an assay technique for soft agar colony formation using MTT, however, insoluble formazan was formed, the addition of a surfactant was required, and assay accuracy was disadvantageously lowered due to foaming caused by the addition of the surfactant. As a result of the addition of an agar solubilization buffer without the removal of the medium and the use of part of a cell suspension in solubilized soft agar, further, an enzymatic reaction system is diluted. This results in lowered reaction efficiency, which may disadvantageously cause fluctuations in data.

### SUMMARY OF THE INVENTION

### Object to be Attained by the Invention

Under the above circumstances, it is an object of the present invention to provide a quantitative assay technique for soft agar colony formation that can be carried out rapidly with high accuracy.

### Means for Attaining the Object

The present inventors have conducted concentrated studies in order to attain the above object. As a result, they discovered that a quantitative assay technique for soft agar colony formation that can be carried out rapidly with high accuracy could be provided with the use of tetrazolium that produces water-soluble formazan without the need for the addition of an agar solubilization buffer or a surfactant. This has led to the completion of the present invention.

The present invention includes the following.

(1) A method for evaluation of cell survival comprising:

a step of overlaying agar at the bottom of a vessel with agar containing cells, overlaying the agar containing cells with a medium and culturing the cells;

a step of removing the medium, adding tetrazolium that produces water-soluble formazan and an electron carrier and culturing the cells; and

a step of evaluating cell survival based on the color developed by the water-soluble formazan.

(2) The method according to (1), wherein the tetrazolium that produces water-soluble formazan is 2-(4-iodophenyl)-3-(4-nitrophenyl)-5-(2,4-disulfophenyl)-2H-tetrazolium monosodium salt.

(3) The method according to (1) or (2), wherein the electron carrier is 1-methoxy-5-methylphenazinium methyl sulfate.

(4) The method according to any one of (1) to (3), wherein the concentration of the agar at the bottom of the vessel is 0.55% to 0.65%.

(5) The method according to any one of (1) to (4), wherein the concentration of the agar containing cells is 0.35% to 0.45%.

(6) A method for screening for an anticancer agent comprising:

a step of overlaying agar at the bottom of a vessel with agar containing cancer cells, overlaying the agar containing cancer cells with a medium and culturing the cancer cells, wherein the agar containing cancer cells or medium contains a candidate anticancer agent;

a step of removing the medium, adding tetrazolium that produces water-soluble formazan and an electron carrier and culturing the cancer cells; and

a step of evaluating cancer cell growth based on the color developed by the water-soluble formazan.

(7) The method according to (6), wherein the tetrazolium that produces water-soluble formazan is 2-(4-iodophetayl)-3-(4-nitrophenyl)-5-(2,4-disulfophenyl)-2H-tetrazolium monosodium salt.

(8) The method according to (6) or (7), wherein the electron carrier is 1-methoxy-5-methylphenazinium methyl sulfate.

(9) The method according to any one of (6) to (8), wherein the concentration of the agar at the bottom of the vessel is 0.55% to 0.65%.

(10) The method according to any one of (6) to (9), wherein the concentration of the agar containing cancer cells is 0.35% to 0.45%.

(11) A kit for evaluation of cell survival or cell growth comprising tetrazolium that produces water-soluble formazan, an electron carrier, and agar.

(12) The kit according to (11), wherein the tetrazolium that produces water-soluble formazan is 2(4-iodophenyl)-3-(4-nitrophen)4)-5-(2,4-disulfophenyl)-2H-tetrazolium monosodium salt.

(13) The kit according to (11) or (12), wherein the electron carrier is 1-methoxy-5-methylphenazinium methyl sulfate.

This description includes part or all of the content as disclosed in the description and/or drawings of Japanese Patent Application No. 2011-219433, which is a priority document of the present application.

### EFFECTS OF THE INVENTION

The present invention provides a quantitative assay technique for soft agar colony formation that can be carried out rapidly with high accuracy. By performing such quantitative assay technique for soft agar colony formation, evaluation of cell survival or screening of an anticancer agent can be carried out with high efficiency.

### BRIEF DESCRIPTION OF THE DRAWINGS

FIG. 1 shows a chart showing the number of cells and the results of quantitative evaluation of the growth of cancer cells (DU145 cells and Panel cells) by the method of the present invention.

FIG. 2 shows a chart showing the concentration-dependent inhibitory effects of Paclitaxel as an anticancer agent on colony formation of cancer cells (DU145 cells) evaluated by the method of the present invention.

FIG. 3 shows a chart showing the concentration-dependent inhibitory effects of a candidate molecule for a novel anticancer agent on colony formation of cancer cells (DU145 cells) evaluated by the method of the present invention.

### EMBODIMENTS FOR CARRYING OUT THE INVENTION

Hereafter, the present invention is described in detail.

The method for evaluation of cell survival according to the present invention (hereafter, referred to as “the method of the invention”) comprises: a step of overlaying agar at the bottom of a vessel with agar containing cells, overlaying the agar containing cells with a medium and culturing the cells; a step of removing the medium, adding tetrazolium that produces water-soluble formazan and an electron carrier and culturing the cells; and a step of evaluating cell survival based on the color developed by the water-soluble formazan. In other words, the method of the invention can be a method for determining the number of cells or a method for evaluating cell growth. According to conventional assay techniques for soft agar colony formation using MTT, such as CytoSelect™ 96-well in vitro tumor sensitivity assay (soft agar colony formation assay; Cell Biolabs, Inc.), insoluble formazan was formed, the addition of a surfactant was required, and assay accuracy was disadvantageously lowered due to foaming caused by the addition of the surfactant. As a result of the addition of an agar solubilization buffer without the removal of the medium, also, an enzymatic reaction system is diluted. This may lower reaction efficiency and cause fluctuations in data, disadvantageously. According to the method of the invention, however, the addition of an agar solubilization buffer and a surfactant is not required, and evaluation of cell survival, determination of the number of cells, or evaluation of cell growth can be performed rapidly with high accuracy.

According to the method of the invention, cell survival is evaluated using tetrazolium that produces water-soluble formazan and an electron carrier based on the color developed, by the water-soluble formazan produced by tetrazolium as a result of reduction by mitochondrial dehydrogenase in a cell upon incorporation into a viable cell. The increased number of viable cells leads to enhanced activity of mitochondrial dehydrogenase in a cell. That is, the correlation between enhanced activity of such enzyme and the production of an increased amount of water-soluble formazan indicates a linear correlation between color development of water-soluble formazan and the number of cells.

Examples of tetrazolium that produces water-soluble formazan used in the method of the present invention include 2-(4-iodophenyl)-3-(4-nitrophenyl)-5-(2,4-disulfophenyl)-2H-tetrazolium monosodium salt (hereafter, referred to as “WST-1”), 2-(2-methoxy-4-nitrophenyl)-3-(4-nitrophenyl)-5-(2,4-disulfophenyl)-2H-tetrazolium monosodium salt (hereafter, referred to as “WST-8”), sodium 3′-[1-phenylaminocarbonyl-3,4-tetrazolium]-bis(4-methoxy-6-nitro)benzene sulfonic acid hydrate (hereafter, referred to as “XTT”), and 3-(4,5-dimethylthiazol-2-yl)-5-(3-carboxymethoxyphenyl)-2-(4-sulfophenyl)-2H-tetrazolium (hereafter, referred to as “MTS”), with WST-1 being preferable.

An electron carrier functions when tetrazolium is converted to water-soluble formazan by dehydrogenase activity. When NADH and NADPH reduced from oxidized nicotinamide adenine dinucleotide (NAD⁺) and oxidized nicotinamide adenine dinucleotide phosphate (NADP⁺) are converted to oxidized forms again by the activity of mitochondrial dehydrogenase in a cell, an electron carrier accepts hydrogen, and it is then converted to the reduced form. When such reduced form is converted again to an electron carrier, tetrazolium is reduced to water-soluble formazan. Examples of electron carriers include 1-methoxy-5-methylphenazinium methyl sulfate (hereafter, referred to as “1-methoxy PMS”) and N-methylphenazinium methyl sulfate (hereafter, referred to as “PMS”), with 1-methoxy PMS being preferable.

The reactions whereby water-soluble formazan is produced when WST-1 and 1-methoxy PMS are used are exemplified below.

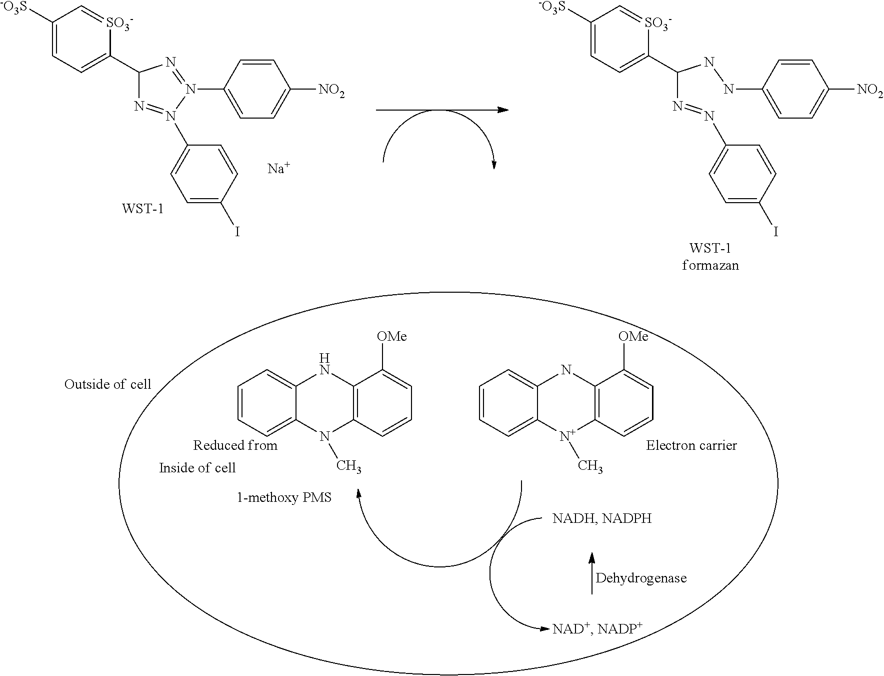

In the reactions shown above, NADH and NADPH reduced from NAD⁺ and NADP⁺ by the activity of mitochondrial dehydrogenase in a cell are converted again to the oxidized forms. In such a case, 1-methoxy PMS accepts hydrogen, and it is converted to the reduced form. Such reduced form reduces WST-1 when it is converted again to an electron carrier, resulting in the production of corresponding water-soluble WST-1 formazan (compound name: 1-(4-iodophenyl)-3-(2,4-disulfophenyl)-5-(4-nitrophenyl)formazan).

Water-soluble formazan produced when WST-8 is used is corresponding water-soluble WST-8 formazan (compound name: 1-(2-methoxy-4-nitrophenyl)-3-(2,4-disulfophenyl)-5-(4-nitrophenyl)formazan). Water-soluble formazan produced when XTT is used is corresponding water-soluble formazan (compound name: 1,5 -bis[(4-methoxy-6-nitro)benzene sulfonic acid]-3-phenylaminocarbonyl-formazan). Water-soluble formazan produced when MTS is used is corresponding water-soluble formazan (compound name; 1-(4-sulfophenyl)3-(3-carboxymethoxyphenyl)-5-(4,5-dimethylthiazol-2-yl)formazan).

In the method of the invention, commercially available tetrazolium and electron carriers can be used.

According to the method of the invention, a culture is first prepared. Specifically, agar at the bottom of a vessel is overlaid with agar containing cells, and the agar containing cells is overlaid with a medium. Examples of vessels include 96-well and 24-well microtiter plates. Concentration of agar to be introduced into a vessel at its bottom (i.e., bottom agar) is, for example, 0.5% to 0.7%, preferably 0.55% to 0.65%, and more preferably 0.6%. In contrast, concentration of agar containing cells (i.e., soft agar) is, for example, 0.35% to 0.45%, and preferably 0.4%.

For example, a 96-well microtiter plate is used as a culture vessel, a medium containing 0.6% agar is added as bottom agar at approximately 60 μl/well, and the agar is allowed to solidify in a refrigerator or the like for approximately 5 minutes. Subsequently, the plate is heated in a CO₂ gas incubator at 37° C.

Separately, cultured cells as specimens are removed, the number thereof is counted, the cells are diluted to realize the cell density of approximately 2,000 to 10,000 cells/well, and the resultant is mixed with a medium containing 0.4% agar. The resulting agar containing cells is introduced into the above-described plate at approximately 75 μl/well, and the agar is allowed to solidify in a refrigerator or the like for approximately 5 minutes. Thereafter, the plate is heated again in a CO₂ gas incubator at 37° C.

Further, a medium is introduced into the above-described plate at approximately 100 μl/well.

Thus, the culture used in the method of the invention can be prepared.

The resulting culture product is cultured for a period that is long enough to develop many colonies (e.g., for a week to 10 days).

After culture was conducted, an upper-layer medium is removed, tetrazolium and an electron carrier are added, and culture (i.e., incubation) is carried out again. Concentration of tetrazolium and an electron carrier to be added can be adequately determined. For example, a solution of 4 to 6 μM, and preferably 5 μM tetrazolium, and a solution of 0.15 to 0.3 mM, and preferably 0.2 mM electron carrier are added. For example, a solution of tetrazolium and a solution of an electron carrier are added to a 96-well microtiter plate at approximately 10 μl/well, respectively. Thereafter, the culture is subjected to incubation in for example, a CO₂ gas incubator at 37° C. for approximately 1 to 4 hours.

As a result of incubation, water-soluble formazan is produced from tetrazolium, and soft agar develops color. In the method of the invention, therefore, evaluation of cell survival, determination of the number of cells, or evaluation of cell growth is carried out based on the color developed by the water-soluble formazan. Specifically, the absorbance of soft agar is measured at a preferable wavelength in order to evaluate the color developed by the water-soluble formazan. For example, WST-1 or WST-8 has a red color, and it develops a yellow color upon production of water-soluble WST-1 formazan or water-soluble WST-8 formazan. Thus, the absorbance of soft agar is measured at OD405 or OD450, which is the adequate wavelength for water-soluble WST-1 formazan or water-soluble WST-8 formazan (control wavelength: OD 650 or higher). A higher intensity of color developed by water-soluble WST-1 formazan or water-soluble WST-8 formazan (i.e., a higher absorbance) indicates a larger number of viable cells.

XTT has a yellow color, and it develops an orange color upon production of water-soluble formazan. Thus, the absorbance of soft agar is measured at OD450 to 500, which is the adequate measurement wavelength for water-soluble formazan produced from XTT (control wavelength: OD 650 or higher).

Further, MTS is colorless, and it develops an orange color upon production of water-soluble formazan. Thus, the absorbance of soft agar is measured at OD490, which is the adequate wavelength for water-soluble formazan produced from MTS (control wavelength: OD 630 to 700).

Subsequently, the measured absorbance can be correlated with the number of analyte cells. For example, the number of cells can be determined based on the measured absorbance in relation to the calibration curve prepared with the use of a known number of cells.

Cell survival can be evaluated based on the number of cells determined in the manner described above. In particular, a significantly greater number of viable cells than that before culture indicates the cell growth. For example, anchorage-independent growth capacity of cancer cells can be evaluated by the method of the invention.

According to the method of the invention, cancer cells are used, and a candidate anticancer agent is added to the agar containing cancer cells or the upper-layer medium. This enables screening for effectiveness of a candidate anticancer agent based on the color developed by the water-soluble formazan. If an intensity of color developed by the water-soluble formazan is significantly lower (i.e., the absorbance is significantly lower) when culture is conducted with the addition of the candidate anticancer agent, compared with the ease in which no candidate anticancer agent is added, for example, the candidate anticancer agent of interest can be evaluated to be effective. Cancer cells may be derived from any type of cancer tissue. Also, primary culture products of cancer cells isolated from cancer patients or cancer cell lines may be used.

The method of the invention described above does not require the addition of a surfactant, treatment steps can be omitted, and lowering of assay accuracy caused by foaming can be prevented, as opposed to conventional assay techniques for soft agar colony formation using MTT. According to the method of the invention, also, the addition of an agar solubilization buffer is not necessary, and an enzymatic reaction system is not diluted, leading to enhanced reaction efficiency. Further, the method of the invention can be performed rapidly with high assay accuracy and thus is effective for processing of many specimens.

Also, the present invention relates to a kit for evaluation of cell survival or cell growth comprising tetrazolium that produces water-soluble formazan, an electron carrier, and agar, which can be used in the method of the invention. The kit can additionally comprise, for example, a vessel such as a microtiter plate, a medium, buffer, and instructions.

### EXAMPLES

Hereafter, the present invention is described in greater detail with reference to the examples, although the technical scope of the present invention is not limited to these examples.

### Example 1

### Number of Cells and Quantitative Evaluation of Growth of Cancer Cells (DU145 Cells and Panel Cells) by the Method of the Invention

### 1-1. Materials and Method

Evaluation was carried out using agar (Agarose type VII, A9045, Sigma), a 96-well microtiter plate (163371, NUNC), and RPMI-1640 medium (Nissui Pharmaceutical Co., Ltd.), and 1-methoxy PMS and WST-1 (Dojindo Laboratories) were used for color development.

RPMI medium containing 10% fetal bovine serum and 0.6% soft agar was added to a 96-well plate at 50 μl well. After the plate was allowed to stand at 4° C. for 5 minutes to solidify agar, the plate was transferred into a CO₂ gas incubator at 37° C. and then subjected to incubation therein for at least 10 minutes.

Subsequently, 75 μl of RPMI medium containing 10% fetal bovine serum and 0.4% soft agar that comprises DU145 cells (human prostate cancer cell lines) was seeded at 10,000, 5,000, 2,500, 1,250, or 625 cells/well, and 75 μl of RPMI medium containing 10% fetal bovine serum and 0.4% soft agar that comprises Panel cells (human pancreatic adenocarcinoma cell lines) was seeded at 20,000, 10,000, 5,000, 2,500, 1,250, 625, or 313 cells/well on top of 0.6% soft agar in the 96-well microtiter plate, respectively. In addition, wells containing a cell-free medium consisting of soft agar of the conditions described above were prepared as negative controls. The plate was immediately allowed to stand at 4° C. for 5 minutes to solidify agar, and the plate was then incubated in a CO₂ gas incubator at 37° C. for 10 minutes.

Thereafter, (A) DU145 cells were overlaid with 100 μl of RPMI medium containing 10% fetal bovine serum or a medium containing 100 μmol of Paclitaxel as an anticancer agent. Separately, (B) Panel cells were overlaid with 100 μl of RPMI medium containing 10% fetal bovine serum or sterile distilled water. RPMI medium containing 10% fetal bovine serum (100 μl) was introduced into negative control wells. Culture was conducted for 7 days, the overlaying medium or distilled water was removed, and agar was overlaid with 7 mmol of a HEPES (pH 7.4) solution containing 5 μmol of WST-1 and 0.2 mmol of 1-methoxy PMS at 12 μl/well.

After incubation was carried out in a CO₂ gas incubator at 37° C. for 2 hours, OD405/OD750 was measured using a microplate reader. All samples were subjected to measurement at 3 different points. The value attained by subtracting the OD value of the negative control well as the background value was designated as the measured value.

### 1-2. Results

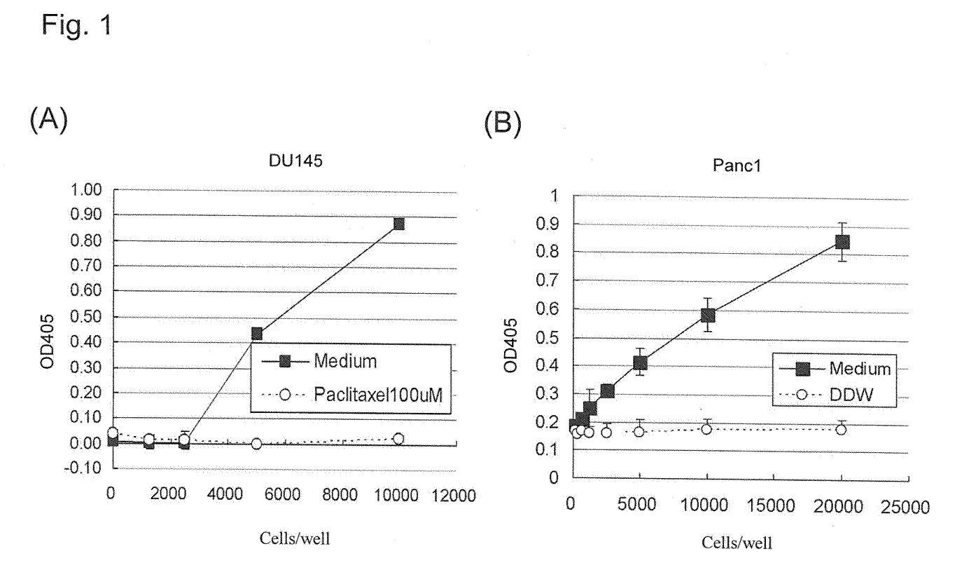

FIG. 1 shows the results. In FIG. 1, a chart in (A) shows the correlation between the absorbance (OD 405) and the number of DU145 cells, and a chart in (B) shows the correlation between the absorbance (OD 405) and the number of Panel cells.

As shown in FIG. 1, OD values correlated with the number of seeded cells were observed when 10,000 or 5,000 DU145 cells were seeded. Also, OD values correlated with the number of seeded cells were observed when 20,000, 10,000, 5,000, 2,500, 1,250, 625, or 313 Panel cells were seeded.

When distilled water or Paclitaxel as an anticancer agent was used, the OD value was low, and it was not correlated with the number of cells. That is such results were apparently different from the ease in which a medium was used.

The significant OD value was not attained when 2,500, 1,250, or 625 DU145 cells were seeded. This indicates that approximately 5,000 DU145 cells are required to achieve colony formation.

### Example 2

### Evaluation of Inhibitory Effects of Paclitaxel as Anticancer Agent on Colony Formation of Cancer Cells (DU145 cells) by the Method of the Invention

### 2-1. Materials and Method

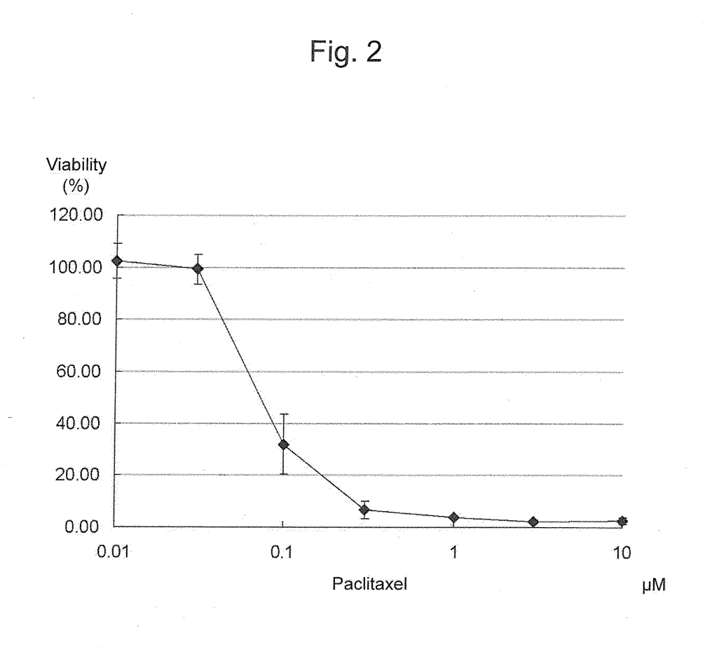

Evaluation was carried out in the same manner as in Section 1-1 of Example 1, except that DU145 cells were seeded at 10,000 cells/well, only the medium was used as a control, and 100 μl of Paclitaxel as an anticancer agent adjusted to the concentration as shown in FIG. 2 was overlaid thereon. Measurement was carried out after culture had been conducted for 1 week, in the same manner as described above.

The OD value of the negative control was subtracted from the OD value at each concentration. This value was divided by the value attained by subtracting the OD value of the negative control from the OD value measured for wells containing no Paclitaxel as an anticancer agent. The resulting value was multiplied by 100, so as to indicate the cell viability.

### 2-2. Results

FIG. 2 shows the results. In FIG. 2, a vertical axis represents viability (%).

As shown in FIG. 2, cell viability was lowered by Paclitaxel as an anticancer agent in a concentration-dependent manner.

### Example 3

### Evaluation of Inhibitory Effects of Novel Chemical Substances A, B, and C on Colony Formation of Cancer Cells (DU145 cells) by the Method of the Invention

### 3-1. Materials and Method

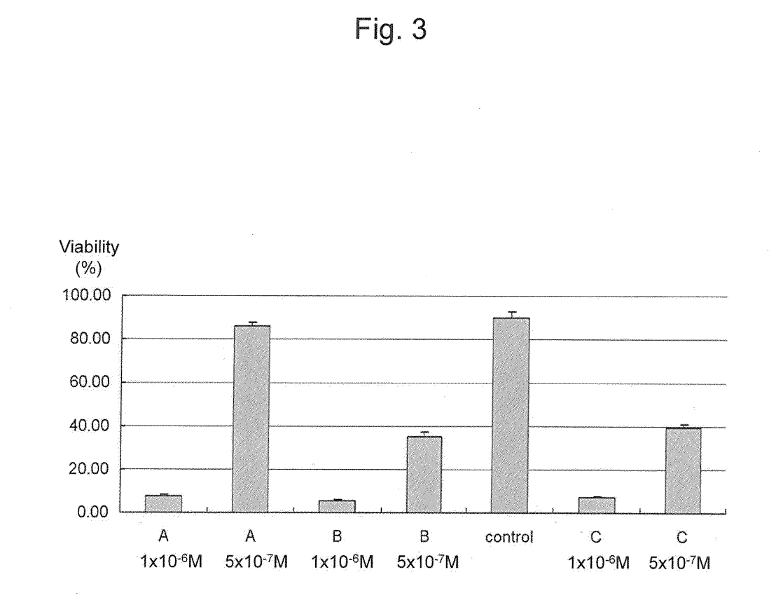

Evaluation was carried out in the same manner as in Section 1-1 of Example 1, except that DU145 cells were seeded at 10,000 cells/well, only the medium was used as a control, and 100 μl of novel chemical substances A, B, and C was overlaid on the DU145 cells and the medium, respectively, at the concentration shown in FIG. 3. Measurement was carried out after culture had been conducted for 1 week, in the same manner as described above. Thereafter, the experiment and the measurement were carried out in the same manner as in Section 2-1 of Example 2.

### 3-2. Results

FIG. 3 shows the results. In FIG. 3, a vertical axis represents viability (%).

As shown in FIG. 3, inhibitory effects of the novel chemical substances A, B, and C on colony formation were found to be superior to those of the control. In addition, inhibitory effects were found to vary in a concentration-dependent manner.

### Example 4

### Evaluation of Inhibitory Effects of Low-Molecular-Weight Compounds on Cancer Cell Colony Formation by the Method of the Invention

In Example 4, 11 types of analogous low-molecular-weight compounds were subjected to evaluation of inhibitory effects on cancer cell colony formation in the same manner as in the case of evaluation of inhibitory effects of chemical substances on cancer cell colony formation described m Example 3. The lox-molecular-weight compounds used may be able to function as ideal antitumor agents that have PCA-1 inhibitory effects, fewer (and less severe) side effects (cytotoxicity), and high tumor-shrinking effects.

While the PCA-1 expression level is high in case of prostate cancer, PCA-1 has been reported as a novel gene (i.e., prostate cancer antigen-1: PCA-1), which is not expressed at high level in case of normal prostate epithelial cells or benign tumors such as prostatic hyperplasia (Abstracts of the 123rd Annual Meeting of Pharmaceutical Society of Japan 4, p. 15, 2003; and Konishi, N. et al., Chit. Cancer Res., Jul. 15, 2005; 11 (14): 5090-7).

A method for diagnosis of prostate cancer based on PCA-1 expression (WO 2006/098464) and an apoptosis accelerator, a cell growth inhibitor, or a preventive and/or therapeutic agent for cancer comprising, as an active ingredient, a compound that inhibits expression or functions of PCA-1 (WO 2007/015587) have been reported. Also the PCA-1 expression level is high in case of pancreatic cancer (JP Patent Publication (Kokai) No. 2011-1286 A) or non-small cell lung cancer gasaki, M. et al., Br. J. Cancer., 2011, 104 (4): 700-6). As a result of inhibition of PCA-1 expression in such cancer cells with the aid of siRNA, inhibitory effects on the growth of prostate cancer cells (JP Patent Publication (Kokai) No 2011-1286 A), pancreatic cancer cells (JP Patent Publication (Kokai) No. 2011-1286 A), and non-small-cell lung cancer cells were found to be significant (Tasaki. M. et al., Br. J. Cancer., 2011, 104 (4): 700-6). Also tumors formed by transplantation of cancer cells into mice were observed to have undergone regression as a result of siRNA administration against PCA-1. These results indicate that PCA-1 can serve as a new molecular target for treatment of cancer, including prostate cancer and pancreatic cancer.

PCA-1 is also referred to as the human AlkB homolog 3 (hALKBH3), and it was found to catalyze demethylation of DNA and RNA in recent years (DNA unwinding by ASCC3 helicase is coupled to ALKBH3-dependent DNA alkylation repair and cancer cell proliferation, Dango, S., Mosammaparast, N., Sowa, M. E., Xiong, L. J., Wu, F., Park, K., Rubin, M., Gygi, S., Harper, J. W., and Shi, Y., Mol. Cell, Nov. 4, 2011; 44 (3): 373-84).

PCA-1 enzyme inhibitory activity can be assayed based on progression of PCR reactions in proportion to the degree of demethylation of the methylated substrate DNA.

PCA-1 enzyme inhibitory activity was evaluated in the manner described below. Test compounds (low-molecular-weight compounds) (10 μM and 1 μM) and 4 ng of silkworm recombinant PCA-1 were added to an enzyme reaction solution containing, as a substrate, 80 fmol of 3-methylcytosine-containing oligo DNA (i.e., 50 mM Tris-HCl buffer (pH 8.0), 2 mM ascorbic acid, 100 μM oxoglutaric acid, and 40 μM iron sulfate), and incubation was carried out at 37° C. for 1 hour. After the completion of the reaction, the enzymatic reaction solution was diluted 20-fold with water, so as to terminate the reaction. Real-time PCR was carried out using 2 μl of the reaction solution in 20 μl of the reaction system (Bio-Rad iQ SYBR Green Supermix.). A calibration curve was prepared using a dilution series of nonmethylated oligo DNA. A 24-base forward primer and a 22-base reverse primer were used, and the reactions were allowed to proceed under the conditions described below: 95° C. for 10 seconds; 40 cycles of 95° for 5 seconds, 61° C. for 30 seconds, and 72° C. for 15 seconds; 95° C. for 1 minute; 55° C. for 1 minute; 55° C. for 10 seconds, with temperature being raised therefrom by 0.5° C.; and 95° for 10 seconds; followed by storage at 25° C.

The results are shown in Table 1 below. The values shown in Table 1 indicate the amount of PCR products decreased in the presence of the test compounds (low-molecular-weight compounds), relative to those in the absence of the test compounds (low-molecular-weight compounds) in percent figures. The values indicate PCA-1 enzyme inhibitory activity of the test compounds (low-molecular-weight compounds). The inhibitory effects of the 11 types of low-molecular-weight compounds having PCA-1 enzyme inhibitory activity, which were selected based on the evaluation described above, on cell survival were examined.

Also, Table 1 shows the results of comparison between DU145 (prostate cancer cells) and Panel (pancreatic cancer cells) in terms of the inhibitory effects of 10 μmol low-molecular weight compounds on cancer cell growth assayed in accordance with the conventional cell growth assay techniques and the 50% inhibitory concentration of the low-molecular weight compounds evaluated by the method of the invention.

Conventional cell growth assay was carried out in the manner described below. Cells were seeded in 90 μl of medium at 5,000 cells/well in a 96-well plate, general monolayer culture was conducted overnight, 10 μl of the test compounds (low-molecular-weight compounds) was added thereto, and culture was conducted for an additional 48 hours. Thereafter, 10 μl of a 1:9 mixture of an aqueous 1-methoxy PMS solution and a WST-1/20 mM 4-(2-hydroxyethyl)-1-piperazineethanesulfonic acid (HEPES) solution (DOJIN) was added, the absorbance at 450 nm was measured 2 hours later, and the control wavelength of 630 nm was employed. The value attained when test compounds were added was divided by the value attained when phosphate buffer was exclusively added. The result indicates the amount of decrease relative to the value attained when phosphate buffer was added instead of the test compounds in percent figures. Thus, the inhibitory effects of the test compounds (low-molecular-weight compounds) on cell survival were evaluated.

TABLE 1

| | PCA-1 enzyme | | Conventional cell growth assay | | | |
|---|---|---|---|---|---|---|
| Low-molecular- | inhibitory activity | | (Inhibitory rate (%)) | | Method of the invention | |
| weight compound | Inhibitory rate (%) | | DU145 | Panc-1 | 50% Inhibitory concentration | |
| (No.) | 10 μM | 1 μM | 10 μM | 10 μM | DU145 (μM) | Panc1 (μM) |
| 1 | 77.0 | 25.0 | 63.4 | 64.8 | 0.36 ± 0.01 | 0.59 ± 0.02 |
| 2 | 49.0 | 12.0 | 71.9 | 68.6 | 0.35 ± 0.11 | 0.39 ± 0.01 |
| 3 | 64.0 | 38.0 | 58.3 | 66.4 | 0.41 ± 0.01 | 0.41 ± 0.02 |
| 4 | 45.0 | 18.0 | 63.9 | 66.1 | 0.37 ± 0.01 | 0.44 ± 0.02 |
| 5 | 27.0 | 2.0 | 48.0 | 50.7 | 0.42 ± 0.02 | 0.63 ± 0.02 |
| 6 | 61.0 | 21.0 | 61.0 | 62.6 | 0.19 ± 0.01 | 0.60 ± 0.01 |
| 7 | 46.0 | −1.0 | 73.1 | 78.3 | 0.39 ± 0.01 | 0.37 ± 0.01 |
| 8 | 73.0 | 50.0 | 67.2 | 53.1 | 0.73 ± 0.01 | 0.40 ± 0.00 |
| 9 | 14.0 | −13.0 | 43.4 | 39.5 | 0.72 ± 0.05 | 0.59 ± 0.01 |
| 10 | 70.0 | 56.0 | 50.4 | 59.4 | 0.83 ± 0.02 | 2.43 ± 0.05 |
| 11 | 53.8 | 65.2 | 43.9 | 42.3 | 0.89 ± 0.01 | 2.31 ± 0.28 |

As shown in Table 1, the concentration of most low-molecular-weight compounds to inhibit cancer cell growth by 50% was approximately 10 μmol or more than 10 μmol according to conventional cell growth assays. According to the method of the invention, however, the concentration of low-molecular-weight compounds to inhibit cancer cell growth by 50% was as low as 0.3 to 1 μmol. Since these low-molecular-weight compounds do not show significant correlations in terms of inhibitory effects on cancer cell growth evaluated by the conventional cell growth assay technique and by the method of the invention, activity of low-molecular-weight compounds evaluated by conventional cell growth assay was found to be different from that evaluated by the method of the invention.

As shown in Table 1, inhibitory effects of low-molecular-weight compounds on cancer cell growth evaluated by the method of the invention involving the use of soft agar, which is similar to in vivo conditions, were approximately ten to several dozen times higher than those evaluated by a conventional cell growth assay technique conducted via general culture techniques. Thus, the method of the invention was found to be superior to conventional techniques in terms of evaluation of inhibitory effects of candidate anticancer agents on cancer cell growth.

### Example 5

### 1 Evaluation of Inhibitory Effects of Imatinib as Anticancer Agent on Cancer Cell Colony Formation by the Method of the Invention

In Example 5, inhibitory effects of a molecular-targeted agent, Imatinib, which has already been used for treatment of malignant neoplasms and evaluated to be effective, on cancer cell colony formation were evaluated in the same manner as in the case of evaluation of inhibitory effects of chemical substances on cancer cell colony formation described in Example 3 using the K562 cells expressing BCR-Abl, as a therapeutic target molecule of Imatinib, and showing therapeutic effects of Imatinib. Since K562 cells are floating cells, disadvantages caused by the growth in soft agar were deduced to be less significant than in the case of epithelial cells.

The results are shown in Table 2.

Table 2 shows the results of comparison of the concentration of Imatinib to inhibit cell growth by 50% (i.e., IC₅₀) in K562 cells by conventional cell growth assays and by the method of the invention. Conventional cell growth assay was carried out in the manner described in Example 4.

TABLE 2

| | IC₅₀ (nM) | Concentration ratio |
|---|---|---|
| Conventional cell growth assay | 61.28 ± 5.31 | |
| Method of the invention | 23.62 ± 6.86 | x 0.38 |

According to the method of the invention, as shown in Table 2, growth inhibitory effects were observed at concentration of 40% or lower, as opposed to conventional cell growth assay techniques. Since an agent would penetrate into agar, the effective concentration is deduced to be half the concentration determined above. That is, sensitivity of the method of the invention was considered to differ from that of the conventional cell growth assay technique by concentration of approximately 20% or lower (that is, sensitivity of the method of the invention is approximately 5 times higher than that attained by the conventional cell growth assay technique).

As described in Examples 4 and 5, an anticancer agent with low cytotoxicity, which could not be found by conventional techniques (i.e., cell growth assays), can be identified by the method of the invention. Further, a primary method for drug discovery of recent years comprises performing a screening method by identifying a target molecule (e.g., PCA-1 enzyme inhibitory activity shown in Table 1). The method of the invention enables random screening of unidentified target molecules of cancer therapy.

All publications, patents, and patent applications cited herein are incorporated herein by reference in their entirety.

1. A method for evaluation of cell survival comprising:
overlaying agar at the bottom of a vessel with agar containing cells, overlaying the agar containing cells with a medium and culturing the cells;
removing the medium, adding tetrazolium that produces water-soluble formazan and an electron carrier and culturing the cells; and
evaluating cell survival based on the color developed by the water-soluble formazan,
wherein solubilization of the agar with addition of an agar solubilization buffer and dissolution of the produced formazan with addition of a surfactant are not carried out.

2. The method according to claim 1, wherein the tetrazolium that produces water-soluble formazan is 2-(4-iodophenyl)-3-(4-nitrophenyl)-5-(2,4-disulfophenyl)-2H-tetrazolium monosodium salt.

3. The method according to claim 1, wherein the electron carrier is 1-methoxy-5-methylphenazinium methyl sulfate.

4. The method according claim 1, wherein the concentration of the agar at the bottom of the vessel is from 0.55% to 0.65%.

5. The method according to claim 1, wherein the concentration of the agar containing cells is from 0.35% to 0.45%.

6. A method for screening for an anticancer agent comprising:
overlaying agar at the bottom of a vessel with agar containing cancer cells, overlaying the agar containing cancer cells with a medium and culturing the cancer cells,
wherein the agar containing cancer cells or medium contains a candidate anticancer agent;
removing the medium, adding tetrazolium that produces water-soluble formazan and an electron carrier and culturing the cancer cells; and
evaluating cancer cell growth based on the color developed by the water-soluble formazan,
wherein solubilization of the agar with addition of an agar solubilization buffer and dissolution of the produced formazan with addition of a surfactant are not carried out.

7. The method according to claim 6, wherein the tetrazolium that produces water-soluble formazan is 2-(4-iodophenyl)-3-(4-nitrophenyl)-5-(2,4-disulfophenyl)-2H-tetrazolium monosodium salt.

8. The method according to claim 6, wherein the electron carrier is 1-methoxy-5-methylphenazinium methyl sulfate.

9. The method according to claim 6, wherein the concentration of the agar at the bottom of the vessel is from 0.55% to 0.65%.

10. The method according to claim 6, wherein the concentration of the agar containing cancer cells is from 0.35% to 0.45%.

11. A kit for evaluation of cell survival or cell growth comprising tetrazolium that produces water-soluble formazan, an electron carrier, and agar, wherein said kit does not comprise an agar solubilization buffer or a surfactant.

12. The kit according to claim 11, wherein the tetrazolium that produces water-soluble formazan is 2-(4-iodophenyl)-3-(4-nitrophenyl)-5-(2,4-disulfophenyl)-2H-tetrazolium monosodium salt.

13. The kit according to claim 11, wherein the electron carrier is 1-methoxy-5-methylphenazinium methyl sulfate.
